(19) United States
(12) Patent Application Publication
(10) Pub. No.: US 2006/0140902 A1
(43) Pub. Date: Jun. 29, 2006

(54) Odor control substrates

(75) Inventors: John Gavin MacDonald, Decatur, GA (US); Roger Bradshaw Quincy III, Cumming, GA (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 11/021,571

(22) Filed: Dec. 23, 2004

(51) Int. Cl. A61L 9/015; A61K 9/70

(52) U.S. Cl. 424/76.2; 424/443; 514/58

(57) ABSTRACT

An odor control substrate that contains a plurality of fibers oriented in the z-direction is provided. At least some of the fibers contain portions that are exposed on an outer surface of the substrate. An odor control coating is applied to the substrate so that a majority of the coating resides on the exposed portions. As a result, the odor control substrate may retain certain of its beneficial properties even after being applied with the odor control coating, such as good extensibility, absorbency, etc.

### BACKGROUND OF THE INVENTION

Odor control additives have been conventionally incorporated into substrates for a variety of reasons. For instance, absorbent articles may contain odor control additives to adsorb compounds that result in the production of malodors contained in absorbed fluids or their degradation products. Examples of these compounds include fatty acids, ammonia, amines, sulfur-containing compounds, ketones and aldehydes. Various types of odor control additives have been employed for this purpose. For instance, activated carbon has been used to reduce a broad spectrum of odors. However, one significant problem associated with many materials that contain activated carbon, particularly those that have a layered structure, is that the materials tend to act as a “barrier” to the efficacy of the activated carbon in reducing odor by preventing the malodors from fully interacting therewith. Even for substrates on which activated carbon is disposed so that it is increasingly exposed to the malodorous environment, its odor control properties often become depleted before use. In addition, activated carbon may also cause a substantial increase in the stiffness of the material, thereby reducing its ability to function in a variety of applications.

As such, a need currently exists for an improved technique for incorporating an odor adsorbent into a substrate.

### SUMMARY OF THE INVENTION

In accordance with one embodiment of the present invention, an odor control substrate is disclosed that has a plane of orientation defined by the cross machine direction and machine direction of the substrate. The substrate comprises a plurality of fibers oriented from about 30° to about 150° relative to an axis that is perpendicular to the plane of orientation, at least a portion of the fibers being exposed on a surface of the substrate. An odor control coating is applied to the substrate so that a majority of the coating resides on the exposed fibers, the odor control coating comprising an odor adsorbent that is capable of adsorbing one or more odorous compounds when contacted therewith.

In accordance with another embodiment of the present invention, a method for forming an odor control substrate is disclosed. The method comprises providing a substrate having a plane of orientation defined by the cross machine direction and machine direction of the substrate. The comprises a plurality of fibers oriented from about 60° to about 120° relative to an axis that is perpendicular to the plane of orientation, at least a portion of the fibers being exposed on a surface of the substrate. A coating formulation is formed that contains activated carbon and a solvent. The surface of the substrate is coated with the formulation and the formulation is dried to form an odor control coating to form an odor control coating that is present on the exposed fibers.

In accordance with still another embodiment of the present invention, a pouch for individually wrapping a feminine care absorbent article is disclosed. The pouch comprises a wrapper that contains a nonwoven fibrous material. The material has a plane of orientation defined by the cross machine direction and machine direction of the material. The nonwoven fibrous material comprises a plurality of fibers oriented from about 30° to about 150° relative to an axis that is perpendicular to the plane of orientation, at least a portion of the fibers being exposed on a surface of the material. An odor control coating is applied to the nonwoven web material so that a majority of the coating resides on the exposed fibers.

Other features and aspects of the present invention are described in more detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figures in which:

FIG. 1 is an illustration of a substrate having a plurality of fibers oriented in the z-direction in accordance with one embodiment of the present invention;

FIG. 2 is an illustration of a substrate having a plurality of fibers oriented in the z-direction in accordance with another embodiment of the present invention;

FIG. 3 is a schematic illustration of a process for coating a substrate in accordance with one embodiment of the present invention;

FIG. 4 is a perspective view of one embodiment of an individually wrapped absorbent article package for use in the present invention;

FIG. 5 is a perspective view of the package of FIG. 4 shown in its opened state;

FIG. 6 is a cross-sectional photograph of one of the samples of Example 1; and

FIG. 7 is a cross-sectional photograph of the sample shown in FIG. 6 at a different section of the sample.

Repeat use of reference characters in the present specification and drawings is intended to represent same or analogous features or elements of the invention.

### DETAILED DESCRIPTION OF REPRESENTATIVE EMBODIMENTS

### Definitions

As used herein, the terms “machine direction” or “MD” generally refers to the direction in which a material is produced. The term “cross-machine direction” or “CD” refers to the direction perpendicular to the machine direction. Dimensions measured in the cross-machine direction are referred to as “width” dimension, while dimensions measured in the machine direction are referred to as “length” dimensions.

As used herein the term “fibers” includes both staple fibers and substantially continuous filaments, unless otherwise indicated. As used herein the term “substantially continuous” with respect to a filament or fiber means a filament or fiber having a length much greater than its diameter, for example having a length to diameter ratio in excess of about 15,000 to 1, and desirably in excess of 50,000 to 1. Likewise, “staple fibers” generally refer to fibers that are natural or cut from a manufactured filament prior to forming into a web. Such staple fibers may sometimes have an average fiber length of from about 1 to about 150 millimeters, in some embodiments from about 5 to about 50 millimeters, in some embodiments from about 10 to about 40 millimeters, and in some embodiments, from about 10 to about 25 millimeters.

As used herein the term “nonwoven fabric or web” refers to a web having a structure of individual fibers or threads that are interlaid, but not in an identifiable manner as in a knitted fabric. Nonwoven fabrics or webs have been formed from many processes such as for example, meltblowing processes, spunbonding processes, bonded carded web processes, etc.

As used herein, the term “meltblown web” generally refers to a nonwoven web that is formed by a process in which a molten thermoplastic material is extruded through a plurality of fine, usually circular, die capillaries as molten fibers into converging high velocity gas (e.g. air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin, et al., which is incorporated herein in its entirety by reference thereto for all purposes. Generally speaking, meltblown fibers may be microfibers that are substantially continuous or discontinuous, generally smaller than 10 microns in diameter, and generally tacky when deposited onto a collecting surface.

As used herein, the term “spunbond web” generally refers to a web containing small diameter substantially continuous fibers. The fibers are formed by extruding a molten thermoplastic material from a plurality of fine, usually circular, capillaries of a spinnerette with the diameter of the extruded fibers then being rapidly reduced as by, for example, eductive drawing and/or other well-known spunbonding mechanisms. The production of spunbond webs is described and illustrated, for example, in U.S. Pat. No. 4,340,563 to Appel, et al., U.S. Pat. No. 3,692,618 to Dorschner, et al., U.S. Pat. No. 3,802,817 to Matsuki, et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Levy, U.S. Pat. No. 3,542,615 to Dobo, et al., and U.S. Pat. No. 5,382,400 to Pike, et al., which are incorporated herein in their entirety by reference thereto for all purposes. Spunbond fibers are generally not tacky when they are deposited onto a collecting surface. Spunbond fibers may sometimes have diameters less than about 40 microns, and are often between about 5 to about 20 microns.

As used herein, the term “multicomponent fibers” generally refers to fibers that have been formed from at least two polymer components. Such fibers are typically extruded from separate extruders, but spun together to form one fiber. The polymers of the respective components are typically different, but may also include separate components of similar or identical polymeric materials. The individual components are typically arranged in substantially constantly positioned distinct zones across the cross-section of the fiber and extend substantially along the entire length of the fiber. The configuration of such fibers may be, for example, a side-by-side arrangement, a pie arrangement, or any other arrangement. Multicomponent fibers and methods of making the same are taught in U.S. Pat. No. 5,108,820 to Kaneko, et al., U.S. Pat. No. 4,795,668 to Kruege, et al., U.S. Pat. No. 5,382,400 to Pike, et al., U.S. Pat. No. 5,336,552 to Strack, et al., and U.S. Pat. No. 6,200,669 to Marmon, et al., which are incorporated herein in their entirety by reference thereto for all purposes. The fibers and individual components containing the same may also have various irregular shapes such as those described in U.S. Pat. No. 5,277,976 to Hogle, et al., U.S. Pat. No. 5,162,074 to Hills, U.S. Pat. No. 5,466,410 to Hills, U.S. Pat. No. 5,069,970 to Largman, et al., and U.S. Pat. No. 5,057,368 to Largman, et al., which are incorporated herein in their entirety by reference thereto for all purposes.

As used herein, the term “extensible” refers to a property of a material or composite by virtue of which it stretches or extends in the direction of an applied biasing force, normally exerted by a consumer, by at least about 25% of its relaxed length. An extensible material may or may not have recovery properties. For example, an elastomeric material is an extendable material having recovery properties. A meltblown web may be extensible, but not have recovery properties.

As used herein, the term “elastomeric” and “elastic” and refers to a material that, upon application of a stretching force, is stretchable in at least one direction (such as the CD direction), and which upon release of the stretching force, contracts/returns to approximately its original dimension. For example, a stretched material may have a stretched length that is at least 50% greater than its relaxed unstretched length, and which will recover to within at least 50% of its stretched length upon release of the stretching force. A hypothetical example would be a one (1) inch sample of a material that is stretchable to at least 1.50 inches and which, upon release of the stretching force, will recover to a length of not more than 1.25 inches. Desirably, such elastomeric sheet contracts or recovers at least 50%, and even more desirably, at least 80% of the stretch length in the cross machine direction.

As used herein, an “absorbent article” refers to any article capable of absorbing water or other fluids. Examples of some absorbent articles include, but are not limited to, personal care absorbent articles, such as diapers, training pants, absorbent underpants, adult incontinence products, feminine hygiene products (e.g., sanitary napkins), swim wear, baby wipes, and so forth; medical absorbent articles, such as garments, fenestration materials, underpads, bandages, absorbent drapes, and medical wipes; food service wipers; clothing articles; and so forth. Materials and processes suitable for forming such absorbent articles are well known to those skilled in the art.

### DETAILED DESCRIPTION

Reference now will be made in detail to various embodiments of the invention, one or more examples of which are set forth below. Each example is provided by way of explanation, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention cover such modifications and variations.

In general, the present invention is directed to an odor control substrate that contains a plurality of fibers oriented in the z-direction, at least some of which contain portions that are exposed on an outer surface of the substrate. An odor control coating is applied to the substrate so that a majority of the coating resides on the exposed fiber portions. As a result, the odor control substrate may retain certain of its beneficial properties even after being applied with the odor control coating, such as good extensibility, absorbency, etc.

A. Substrates

Any of variety of substrates may be coated with the odor control coating in accordance with the present invention. For example, nonwoven webs, woven fabrics, knit fabrics, and so forth, may be coated with the odor control coating. In most embodiments, the substrate contains at least one nonwoven web. When utilized, the nonwoven web may include, but not limited to, spunbond webs, meltblown webs, bonded carded webs, air-laid webs, coform webs, hydraulically entangled webs, and so forth. Nonwoven webs may be formed by a variety of different materials. For instance, suitable polymers for forming nonwoven webs may include polyolefins, polyamides, polyesters, polycarbonates, polystyrenes, thermoplastic elastomers, fluoropolymers, vinyl polymers, and blends and copolymers thereof. Suitable polyolefins include, but are not limited to, polyethylene, polypropylene, polybutylene, and so forth; suitable polyamides include, but are not limited to, nylon 6, nylon 6/6, nylon 10, nylon 12 and so forth; and suitable polyesters include, but are not limited to, polyethylene terephthalate, polybutylene terephthalate, polytrimethyl terephthalate, polylactic acid, and so forth. Particularly suitable polymers for use in the present invention are polyolefins including polyethylene, for example, linear low density polyethylene, low density polyethylene, medium density polyethylene, and high density polyethylene; polypropylene; polybutylene; as well as copolymers and blends thereof.

The fibers used to form the nonwoven web may be in the form of substantially continuous fibers, staple fibers, and so forth. Substantially continuous fibers, for example, may be produced by known nonwoven extrusion processes, such as, for example, known solvent spinning or melt-spinning processes. In one embodiment, the nonwoven web contains substantially continuous melt-spun fibers formed by a spunbond process. The spunbond fibers may be formed from any melt-spinnable polymer, co-polymers or blends thereof. The denier of the fibers used to form the nonwoven web may also vary. For instance, in one particular embodiment, the denier of polyolefin fibers used to form the nonwoven web is less than about 6, in some embodiments less than about 3, and in some embodiments, from about 1 to about 3.

In one particular embodiment of the present invention, multicomponent (e.g., bicomponent) fibers are utilized. For example, suitable configurations for the multicomponent fibers include side-by-side configurations and sheath-core configurations, and suitable sheath-core configurations include eccentric sheath-core and concentric sheath-core configurations. In some embodiments, as is well known in the art, the polymers used to form the multicomponent fibers have sufficiently different melting points to form different crystallization and/or solidification properties. The multicomponent fibers may have from about 20% to about 80%, and in some embodiments, from about 40% to about 60% by weight of the low melting polymer. Further, the multicomponent fibers may have from about 80% to about 20%, and in some embodiments, from about 60% to about 40%, by weight of the high melting polymer.

Regardless of the materials, layers, etc., used to form the substrate, it is generally desired that the substrate contain a plurality of fibers oriented in the z-direction. As used herein, the term “z-direction” refers to fibers disposed outside of the plane of orientation of the substrate. For example, the plane of orientation of a substrate is generally a plane defined by the cross machine direction (or the “y-axis”) and machine direction (or the “x-axis”). The z-directional fibers are generally oriented from about 30° to about 150°, in some embodiments from about 60° to about 120°, and in some embodiments, from about 80° to about 100° relative to an axis that is perpendicular to the plane of orientation of the substrate. Such a z-direction fiber orientation may be inherent in the nature of the substrate, or it may be imparted to the substrate through one or more processing steps.

For example, some or all of the fibers of the substrate may be crimped, either mechanically or naturally during fiber formation, to increase fiber orientation in the z-direction. A naturally crimped fiber is a fiber that is crimped by activating a latent crimp contained in the fiber. Crimping techniques are generally well known in the art. For example, U.S. Pat. Nos. 3,595,731 and 3,423,266 to Davies et al., which are incorporated herein in their entirety by reference thereto for all purposes, describe techniques for mechanically crimping fibers. Likewise, U.S. Pat. No. 4,068,036 to Stanistreet and U.S. Pat. No. 5,382,400 to Pike, et al., which are incorporated herein in their entirety by reference thereto for all purposes, describe techniques for inducing fiber crimp using heat treatment. U.S. Pat. No. 6,054,002 to Griesbach, et al., which is incorporated herein in its entirety by reference thereto for all purposes, describes a method of forming self-crimping multicomponent spunbond fibers utilizing a polyolefin component and a non-polyurethane elastomeric block copolymer component, such as copolyesters, polyamide polyether block copolymers and A-B or A-B-A block copolymers with a styrenic moiety. These fibers are crimped by simply drawing the molten fibers, and thereafter releasing the attenuating force. Further, U.S. Pat. No. 5,876,840 to Ning, et al., which is incorporated herein in its entirety by reference thereto for all purposes, describes spunbond multicomponent fibers having a non-ionic surfactant additive within one of the components to accelerate its solidification rate. By adding the non-ionic surfactant to one of the components of the multicomponent fiber, a latent crimp is activated by drawing unheated air. Still other suitable crimping techniques that may be used in the present invention are described in U.S. Pat. No. 3,589,956 to Kranz, et al.; U.S. Pat. No. 5,336,552 to Strack, et al.; and U.S. Pat. No. 6,454,989 to Neely, et al., which are incorporated herein in their entirety by reference thereto for all purposes. Regardless of the technique utilized, the degree of crimping (as measured by “crimps per inch (cpi)”) may generally vary, such as from about 3 to about 100 cpi, in some embodiments from about 5 to about 25 cpi, and in some embodiments, from about 5 to about 15 cpi.

Apart from crimping, various other known techniques also exist for forming a substrate with a plurality of fibers oriented in the z-direction. In some embodiments, staple fibers may be incorporated into the substrate in such a manner that they become oriented in the z-direction. For instance, U.S. Pat. No. 4,590,114 to Holtman and U.S. Pat. No. 4,837,067 to Carey, Jr. et al., which are incorporated herein in their entirety by reference thereto for all purposes, describe various techniques for forming such a nonwoven web using staple fibers.

Still another suitable technique for forming a substrate with a plurality of fibers oriented in the z-direction involves creping. Creping techniques are well known in the art, and may include, for instance, a process by which a wet web is carried over a drum surface, heated, and removed from the drum surface by a creping blade. The blade causes those portions of the web adhered to the drum surface to become more oriented in the z-direction. In addition to or in lieu of wet creping, the substrate may be subjected to a dry creping process (e.g., single recreping (SRC), double recreping (DRC), etc.). Some suitable dry creping techniques are described in U.S. Pat. No. 3,879,257 to Gentile, et al.; U.S. Pat. No. 6,315,864 to Anderson, et al.; and U.S. Pat. No. 6,500,289 to Merker, et al., which are incorporated herein in their entirety by reference thereto for all purposes. Of course, other creping techniques may also be utilized in the present invention. For example, in some embodiments, the substrate may be creped using a “microcreping” process. Some suitable microcreping processes are described in U.S. Pat. No. 3,260,778 to Walton; U.S. Pat. No. 4,919,877 to Parsons, et al.; U.S. Pat. No. 5,102,606 to Ake, et al.; U.S. Pat. No. 5,498,232 to Scholz; and U.S. Pat. No. 5,972,039 to Honeycutt, et al., which are all incorporated herein in their entirety by reference thereto for all purposes. Commercially available microcreping equipment may be obtained from Micrex Corporation of Walpole, Mass.

When oriented in the z-direction, at least a portion of the fibers generally become exposed on a surface of the substrate. As will be discussed in more detail below, a majority (i.e., about 50 wt. %) of the odor control coating is typically present on such exposed portions to limit any adverse affect the coating might otherwise have on certain properties of the substrate, such as its flexibility, absorbency, and so forth. In some embodiments, for example, the substrate remains generally extensible in one or more directions (e.g., x-, y-, and/or z-directions) after being applied with the coating, which means that the substrate may stretch or extend in a direction of an applied biasing force by at least about 25% of its relaxed length. To further ensure that the substrate remains extensible, the substrate may be subjected to various processes to further improve extensibility, such as aperturing, neck-stretching, heat activation, embossing, micro-straining, etc. Likewise, certain materials may also be incorporated into the substrate to improve extensibility. For example, in one embodiment, the substrate may contain an elastic material that is not only extensible, but also has recovery properties. The elastic material may form the entire substrate or simply a portion of the substrate. For instance, elastic strands or sections may be uniformly or randomly distributed throughout the substrate. Alternatively, an elastic film and/or an elastic nonwoven web may be incorporated as a layer of the substrate.

Any material that possesses elastic characteristics may be used in the elastic material. For example, an elastomeric polymer may be utilized, such as elastomeric polyesters, elastomeric polyurethanes, elastomeric polyamides, elastomeric polyolefins, elastomeric copolymers, and so forth. Examples of elastomeric copolymers include block copolymers having the general formula A-B-A′ or A-B, wherein A and A′ are each a thermoplastic polymer endblock that contains a styrenic moiety and B is an elastomeric polymer midblock, such as a conjugated diene or a lower alkene polymer. Such copolymers may include, for instance, styrene-isoprene-styrene (S—I—S), styrene-butadiene-styrene (S—B—S), styrene-ethylene-butylene-styrene (S-EB-S), styrene-isoprene (S—I), styrene-butadiene (S—B), and so forth. Commercially available A-B-A′ and A-B-A-B copolymers include several different S-EB-S formulations from Kraton Polymers of Houston, Tex. under the trade designation KRATON®). KRATON® block copolymers are available in several different formulations, a number of which are identified in U.S. Pat. Nos. 4,663,220, 4,323,534, 4,834,738, 5,093,422 and 5,304,599, which are hereby incorporated in their entirety by reference thereto for all purposes. Other commercially available block copolymers include the S-EP-S elastomeric copolymers available from Kuraray Company, Ltd. of Okayama, Japan, under the trade designation SEPTON®). Still other suitable copolymers include the S—I—S and S—B—S elastomeric copolymers available from Dexco Polymers of Houston, Tex. under the trade designation VECTOR®. Also suitable are polymers composed of an A-B-A-B tetrablock copolymer, such as discussed in U.S. Pat. No. 5,332,613 to Taylor, et al., which is incorporated herein in its entirety by reference thereto for all purposes. An example of such a tetrablock copolymer is a styrene-poly(ethylene-propylene)-styrene-poly(ethylene-propylene) (“S-EP-S-EP”) block copolymer.

Examples of elastomeric polyolefins include ultra-low density elastomeric polypropylenes and polyethylenes, such as those produced by “single-site” or “metallocene” catalysis methods. Such elastomeric olefin polymers are commercially available from ExxonMobil Chemical Co. of Houston, Tex. under the trade designations ACHIEVE® (propylene-based), EXACT® (ethylene-based), and EXCEED® (ethylene-based). Elastomeric olefin polymers are also commercially available from DuPont Dow Elastomers, LLC (a joint venture between DuPont and the Dow Chemical Co.) under the trade designation ENGAGE® (ethylene-based) and from Dow Chemical Co. of Midland, Mich. under the name AFFINITY® (ethylene-based). Examples of such polymers are also described in U.S. Pat. Nos. 5,278,272 and 5,272,236 to Lai, et al., which are incorporated herein in their entirety by reference thereto for all purposes. Also useful are certain elastomeric polypropylenes, such as described in U.S. Pat. No. 5,539,056 to Yang, et al. and U.S. Pat. No. 5,596,052 to Resconi, et al., which are incorporated herein in their entirety by reference thereto for all purposes.

If desired, blends of two or more polymers may also be utilized. For example, a blend of a high performance elastomer and a lower performance elastomer may be utilized. A high performance elastomer is generally an elastomer having a low level of hysteresis, such as less than about 75%, and in some embodiments, less than about 60%. Likewise, a low performance elastomer is generally an elastomer having a high level of hysteresis, such as greater than about 75%. Particularly suitable high performance elastomers may include styrenic-based block copolymers, such as described above and commercially available from Kraton Polymers under the trade designation KRATON®) and from Dexco Polymers under the trade designation VECTOR®. Likewise, particularly suitable low performance elastomers include elastomeric polyolefins, such as metallocene-catalyzed polyolefins (e.g., single site metallocene-catalyzed linear low density polyethylene) commercially available from Dow Chemical Co. under the trade designation AFFINITY®. In some embodiments, the high performance elastomer may constitute from about 25 wt. % to about 90 wt. % of the blend, and the low performance elastomer may likewise constitute from about 10 wt. % to about 75 wt. % of the blend. Further examples of such a high performance/low performance elastomer blend are described in U.S. Pat. No. 6,794,024 to Walton, et al., which is incorporated herein in its entirety by reference thereto for all purposes.

It is sometimes desired that the elastic material be contained within an elastic laminate that also contains one or more other layers, such as foams, films, apertured films, and/or nonwoven webs. An elastic laminate generally contains layers that may be bonded together so that at least one of the layers has the characteristics of an elastomeric polymer. The elastic material used in the elastic laminates may be made from materials, such as described above, that are formed into films, such as a microporous film; fibrous webs, such as a web made from meltblown or spunbond fibers; foams; and so forth.

One example of a suitable elastic nonwoven laminate includes, for instance, a “neck-bonded” laminate. A “neck-bonded” laminate refers to a composite material having at least two layers in which one layer is a necked, non-elastic layer and the other layer is an elastic layer. The resulting laminate is thereby a material that is elastic in the cross-direction and/or machine-direction. Some examples of neck-bonded laminates are described in U.S. Pat. Nos. 5,226,992, 4,981,747, 4,965,122, and 5,336,545, all to Morman, all of which are incorporated herein in their entirety by reference thereto for all purposes.

Another suitable elastic nonwoven laminate may be a “stretch-bonded” laminate, which refers to a composite material having at least two layers in which one layer is a gatherable layer and in which the other layer is an elastic layer. The layers are joined together when the elastic layer is in an extended condition so that upon relaxing the layers, the gatherable layer is gathered. For example, one elastic member may be bonded to another member while the elastic member is extended at least about 25 percent of its relaxed length. Such a multilayer composite elastic material may be stretched until the nonelastic layer is fully extended. One suitable type of stretch-bonded laminate is a spunbond laminate, such as disclosed in U.S. Pat. No. 4,720,415 to VanderWielen et al., which is incorporated herein in its entirety by reference thereto for all purposes. Another suitable type of stretch-bonded laminate is a continuous filament spunbond laminate, such as disclosed in U.S. Pat. No. 5,385,775 to Wright, which is incorporated herein in its entirety by reference thereto for all purposes. For instance, Wright discloses a composite elastic material that includes: (1) an anisotropic elastic fibrous web having at least one layer of elastic meltblown fibers and at least one layer of elastic fibers autogenously bonded to at least a portion of the elastic meltblown fibers, and (2) at least one gatherable layer joined at spaced-apart locations to the anisotropic elastic fibrous web so that the gatherable layer is gathered between the spaced-apart locations. The gatherable layer is joined to the elastic fibrous web when the elastic web is in a stretched condition so that when the elastic web relaxes, the gatherable layer gathers between the spaced-apart bonding locations. Other composite elastic materials are described and disclosed in U.S. Pat. No. 4,789,699 to Kieffer et al., U.S. Pat. No. 4,781,966 to Taylor, U.S. Pat. No. 4,657,802 to Morman, and U.S. Pat. No. 4,655,760 to Morman et al., all of which are incorporated herein in their entirety by reference thereto for all purposes.

A “necked stretch bonded” laminate may also be utilized. As used herein, a necked stretch bonded laminate is defined as a laminate made from the combination of a neck-bonded laminate and a stretch-bonded laminate. Examples of necked stretch bonded laminates are disclosed in U.S. Pat. No. 5,114,781 and 5,116,662, which are both incorporated herein in their entirety by reference thereto for all purposes. Of particular advantage, a necked stretch bonded laminate may be stretchable in both the machine and cross-machine directions.

In addition to the materials described above, the substrate may also contain various other layers and/or materials. For example, the substrate may contain a film that is bonded to the fibrous component of the substrate using well-known techniques. Some suitable techniques for bonding a film to a fibrous web are described in U.S. Pat. No. 5,843,057 to McCormack; U.S. Pat. No. 5,855,999 to McCormack; U.S. Pat. No. 6,002,064 to Kobylivker, et al.; U.S. Pat. No. 6,037,281 to Mathis, et al.; and U.S. Pat. No. 6,402,265 to Jones, et al., which are incorporated herein in their entirety by reference thereto for all purposes. To form a film, a variety of materials may be utilized. For instance, some suitable thermoplastic polymers used in the fabrication of films may include, but are not limited to, polyolefins (e.g., polyethylene, polypropylene, etc.), including homopolymers, copolymers, terpolymers and blends thereof; ethylene vinyl acetate; ethylene ethyl acrylate; ethylene acrylic acid; ethylene methyl acrylate; ethylene normal butyl acrylate; polyurethane; poly(ether-ester); poly(amid-ether) block copolymers; and so forth. In one particular embodiment, the film may be made a liquid-impermeable plastic film, such as a polyethylene and polypropylene film. Generally, such plastic films are impermeable to gases and water vapor, as well as liquids. In addition, the film may be impermeable to liquids, but permeable to gases and water vapor (i.e., “breathable”).

B. Odor Control Coating

In accordance with the present invention, the odor control coating contains an odor adsorbent for adsorbing one or more odorous compounds. Some examples of suitable odor adsorbents include, but are not limited to, activated carbon, zeolites, silica, clays (e.g., smectite clay), alumina, magnesia, titania, cyclodextrins, combinations thereof, and so forth. For instance, activated carbon may be derived from a variety of sources, such as from sawdust, wood, charcoal, peat, lignite, bituminous coal, coconut shells, etc. Some suitable forms of activated carbon and techniques for formation thereof are described in U.S. Pat. No. 5,693,385 to Parks; U.S. Pat. No. 5,834,114 to Economy, et al.; U.S. Pat. No. 6,517,906 to Economy, et al.; U.S. Pat. No. 6,573,212 to McCrae, et al., as well as U.S. Patent Application Publication Nos. 2002/0141961 to Falat, et al. and 2004/0166248 to Hu, et al., all of which are incorporated herein in their entirety by reference thereto for all purposes. Regardless, the concentration of the odor adsorbent is generally tailored to facilitate odor control without adversely affecting other properties of the substrate. For instance, the odor adsorbent may be present in the coating (prior to drying) in an amount from about 1 wt. % to about 50 wt. %, in some embodiments from about 5 wt. % to about 25 wt. %, and in some embodiments, from about 10 wt. % to about 20 wt. %.

If desired, the odor control coating may also contain a binder for increasing the durability of the odor adsorbent when applied to a substrate, even when present at high levels. The binder may also serve as an adhesive for bonding one substrate to another substrate. Generally speaking, any of a variety of binders may be used in the odor control coating of the present invention. Suitable binders may include, for instance, those that become insoluble in water upon crosslinking. Crosslinking may be achieved in a variety of ways, including by reaction of the binder with a polyfunctional crosslinking agent. Examples of such crosslinking agents include, but are not limited to, dimethylol urea melamine-formaldehyde, urea-formaldehyde, polyamide epichlorohydrin, etc.

In some embodiments, a polymer latex may be employed as the binder. The polymer suitable for use in the lattices typically has a glass transition temperature of about 30° C. or less so that the flexibility of the resulting substrate is not substantially restricted. Moreover, the polymer also typically has a glass transition temperature of about −25° C. or more to minimize the tackiness of the polymer latex. For instance, in some embodiments, the polymer has a glass transition temperature from about −15° C. to about 15° C., and in some embodiments, from about −10° C. to about 0° C. For instance, some suitable polymer lattices that may be utilized in the present invention may be based on polymers such as, but are not limited to, styrene-butadiene copolymers, polyvinyl acetate homopolymers, vinyl-acetate ethylene copolymers, vinyl-acetate acrylic copolymers, ethylene-vinyl chloride copolymers, ethylene-vinyl chloride-vinyl acetate terpolymers, acrylic polyvinyl chloride polymers, acrylic polymers, nitrile polymers, and any other suitable anionic polymer latex polymers known in the art. The charge of the polymer lattices described above may be readily varied, as is well known in the art, by utilizing a stabilizing agent having the desired charge during preparation of the polymer latex. For instance, specific techniques for an activated carbon/polymer latex system are described in more detail in U.S. Pat. No. 6,573,212 to McCrae, et al. Commercially available activated carbon/polymer latex systems that may be used in the present invention include Nuchar® PMA, DPX-8433-68A, and DPX-8433-68B, all of which are available from MeadWestvaco Corp of Covington, Va.

Although polymer lattices may be effectively used as binders in the present invention, such compounds sometimes result in a reduction in drapability and an increase in residual odor. Thus, the present inventors have discovered that water-soluble organic polymers may also be employed as binders to alleviate such concerns. Another benefit of the water-soluble binder of the present invention is that it may facilitate the controlled release of the odor control coating from the substrate in an aqueous environment. Specifically, upon contacting an aqueous solution, the water-soluble binder dissolves and loses some of its binding qualities, thereby allowing other components of the odor control coating to be released from the substrate. This may be useful in various applications, such as for hard-surface wipers in which it is desired for the odor control coating to be released into the wiped environment for sustained odor control.

One class of water-soluble organic polymers found to be suitable in the present invention is polysaccharides and derivatives thereof. Polysaccharides are polymers containing repeated carbohydrate units, which may be cationic, anionic, nonionic, and/or amphoteric. In one particular embodiment, the polysaccharide is a nonionic, cationic, anionic, and/or amphoteric cellulosic ether. Suitable nonionic cellulosic ethers may include, but are not limited to, alkyl cellulose ethers, such as methyl cellulose and ethyl cellulose; hydroxyalkyl cellulose ethers, such as hydroxyethyl cellulose, hydroxypropyl cellulose, hydroxypropyl hydroxybutyl cellulose, hydroxyethyl hydroxypropyl cellulose, hydroxyethyl hydroxybutyl cellulose and hydroxyethyl hydroxypropyl hydroxybutyl cellulose; alkyl hydroxyalkyl cellulose ethers, such as methyl hydroxyethyl cellulose, methyl hydroxypropyl cellulose, ethyl hydroxyethyl cellulose, ethyl hydroxypropyl cellulose, methyl ethyl hydroxyethyl cellulose and methyl ethyl hydroxypropyl cellulose; and so forth.

Suitable cellulosic ethers may include, for instance, those available from Akzo Nobel of Covington, Va. under the name “BERMOCOLL.” Still other suitable cellulosic ethers are those available from Shin-Etsu Chemical Co., Ltd. of Tokyo, Japan under the name “METOLOSE”, including METOLOSE Type SM (methycellulose), METOLOSE Type SH (hydroxypropylmethyl cellulose), and METOLOSE Type SE (hydroxyethylmethyl cellulose). One particular example of a suitable nonionic cellulosic ether is ethyl hydroxyethyl cellulose having a degree of ethyl substitution (DS) of 0.8 to 1.3 and a molar substitution (MS) of hydroxyethyl of 1.9 to 2.9. The degree of ethyl substitution represents the average number of hydroxyl groups present on each anhydroglucose unit that have been reacted, which may vary between 0 and 3. The molar substitution represents the average number of hydroxethyl groups that have reacted with each anhydroglucose unit. One such cellulosic ether is BERMOCOLL E 230FQ, which is an ethyl hydroxyethyl cellulose commercially available from Akzo Nobel. Other suitable cellulosic ethers are also available from Hercules, Inc. of Wilmington, Del. under the name “CULMINAL.”

The total concentration of the binders may generally vary depending on the desired properties of the resulting substrate. For instance, high total binder concentrations may provide better physical properties for the coated substrate, but may likewise have an adverse affect on other properties, such as the absorptive capacity or extensibility of the substrate to which it is applied. Conversely, low total binder concentrations may not provide the desired degree of durability. Thus, in most embodiments, the total amount of binder employed in the odor control coating (prior to drying) is from about 0.01 wt. % to about 30 wt. %, in some embodiments from about 0.1 wt. % to about 20 wt. %, and in some embodiments, from about 1 wt. % to about 15 wt. %.

Besides the above-mentioned components, a masking agent may also be employed in the odor control coating to further alter the aesthetic properties of the substrate. That is, the masking agent may enhance opacity and/or alter the color to the coating. To provide optimum masking effects, the size of the particles is desirably less than the size of any odor adsorbent particles employed. For example, the masking particles may have a size less than about 100 micrometers, in some embodiments less than about 50 micrometers, and in some embodiments, less than about 25 micrometers. For example, activated carbon particles may sometimes have a particle size of approximately 35 micrometers. In such cases, the size of the masking particles is typically less than 35 micrometers, and preferably much smaller, such as less than about 10 micrometers. Likewise, the particles may be porous. Without intending to be limited by theory, it is believed that porous particles may provide a passage for odorous compounds to better contact the odor adsorbent. For example, the particles may have pores/channels with a mean diameter of greater than about 5 angstroms, in some embodiments greater than about 20 angstroms, and in some embodiments, greater than about 50 angstroms. The surface area of such particles may also be greater than about 15 square meters per gram, in some embodiments greater than about 25 square meters per gram, and in some embodiments, greater than about 50 square meters per gram. Surface area may be determined by the physical gas adsorption (B.E.T.) method of Bruanauer, Emmet, and Teller, Journal of American Chemical Society, Vol. 60, 1938, p. 309, with nitrogen as the adsorption gas.

In one particular embodiment, porous carbonate particles (e.g., calcium carbonate) are used to alter the black color normally associated with activated carbon odor adsorbents. Such a color change may be more aesthetically pleasing to a user, particularly when the coating is employed on substrates designed for consumer/personal use. Suitable white calcium carbonate particles are commercially available from Omya, Inc. of Proctor, Vt. Still other suitable particles include, but are not limited to, silicates, such as calcium silicate, alumina silicates (e.g., mica powder, clay, etc.), magnesium silicates (e.g., talc), quartzite, calcium silicate fluorite, etc.; alumina; silica; and so forth. The concentration of the particles may generally vary depending on the nature of the particles, and the desired extent of odor control and color alteration. For instance, the particles may be present in the coating (prior to drying) in an amount from about 0.01 wt. % to about 30 wt. %, in some embodiments from about 0.1 wt. % to about 20 wt. %, and in some embodiments, from about 1 wt. % to about 15 wt. %.

Other masking agents may also be employed in the odor control coating. For example, the odor control coating may include a colorant, such as a pigment, dye, etc. The colorant may constitute from about 0.01 to about 20 wt. %, in some embodiments from about 0.1 wt. % to about 10 wt. %, and in some embodiments, from about 0.5 wt. % to about 5 wt. % of the coating. For example, the colorant may be an inorganic and/or organic pigment. Some examples of commercially available organic pigments that may be used in the present invention include those that are available from Clariant Corp. of Charlotte, N.C., under the trade designations GRAPHTOL® or CARTAREN®. Other pigments, such as lake compounds (blue lake, red lake, yellow lake, etc.), may also be employed. Inorganic and/or organic dyes may also be utilized as a colorant. Exemplary organic dye classes include triarylmethyl dyes, monoazo dyes, thiazine dyes, oxazine dyes, naphthalimide dyes, azine dyes, cyanine dyes, indigo dyes, coumarin dyes, benzimidazole dyes, paraquinoidal dyes, fluorescein dyes, diazonium salt dyes, azoic diazo dyes, phenylenediamine dyes, diazo dyes, anthraquinone dyes, trisazo dyes, xanthene dyes, proflavine dyes, sulfonaphthalein dyes, phthalocyanine dyes, carotenoid dyes, carminic acid dyes, azure dyes, acridine dyes, and so forth. One particularly suitable class of dyes includes anthraquinone compounds, which may be classified for identification by their Color Index (CI) number. For instance, some suitable anthraquinones that may be used in the present invention, as classified by their “CI” number, include Acid Black 48, Acid Blue 25 (D&C Green No. 5), Acid Blue 40, Acid Blue 41, Acid Blue 45, Acid Blue 129, Acid Green 25, Acid Green 27, Acid Green 41, Mordant Red 11 (Alizarin), Mordant Black 13 (Alizarin Blue Black B), Mordant Red 3 (Alizarin Red S), Mordant Violet 5 (Alizarin Violet 3R), Natural Red 4 (Carminic Acid), Disperse Blue 1, Disperse Blue 3, Disperse Blue 14, Natural Red 16 (Purpurin), Natural Red 8, Reactive Blue 2, and so forth.

Still other compounds, such as surfactants, electrolytic salts, pH adjusters, etc., may also be included in the odor control coating of the present invention. Although not required, such additional components typically constitute less than about 5 wt. %, in some embodiments less than about 2 wt. %, and in some embodiments, from about 0.001 wt. % to about 1 wt. % of the odor control coating (prior to drying). For example, as is well known in the art, an electrolytic salt may be employed to control the gelation temperature of a water-soluble binder. Suitable electrolytic salts may include, but are not limited to, alkali halides or sulfates, such as sodium chloride, potassium chloride, etc.; alkaline halides or sulfates, such as calcium chloride, magnesium chloride, etc., and so forth.

C. Application of Coatinq

To apply the odor control coating of the present invention to a substrate, the components are first typically dissolved or dispersed in a solvent. For example, one or more of the above-mentioned components may be mixed with a solvent, either sequentially or simultaneously, to form a coating formulation that may be easily applied to a substrate. Any solvent capable of dispersing or dissolving the components is suitable, for example water; alcohols such as ethanol or methanol; dimethylformamide; dimethyl sulfoxide; hydrocarbons such as pentane, butane, heptane, hexane, toluene and xylene; ethers such as diethyl ether and tetrahydrofuran; ketones and aldehydes such as acetone and methyl ethyl ketone; acids such as acetic acid and formic acid; and halogenated solvents such as dichloromethane and carbon tetrachloride; as well as mixtures thereof. The concentration of solvent in the coating formulation is generally high enough to allow easy application, handling, etc. If the amount of solvent is too large, however, the amount of odor adsorbent deposited on the substrate might be too low to provide the desired odor reduction. Although the actual concentration of solvent employed will generally depend on the type of odor adsorbent and the substrate on which it is applied, it is nonetheless typically present in an amount from about 40 wt. % to about 99 wt. %, in some embodiments from about 50 wt. % to about 95 wt. %, and in some embodiments, from about 60 wt. % to about 90 wt. % of the coating formulation.

The solids content and/or viscosity of the coating formulation may be varied to achieve the extent of odor reduction desired. For example, the coating formulation may have a solids content of from about 5% to about 90%, in some embodiments from about 10% to about 80%, and in some embodiments, from about 20% to about 70%. By varying the solids content of the formulation, the presence of the odor adsorbent and other components in the coating formulation may be controlled. For example, to form a coating formulation with a higher level of odor adsorbent, the formulation may be provided with a relatively high solids content so that a greater percentage of odor adsorbent is incorporated into the formulation during the application process. Generally, the viscosity is less than about 2×10⁶ centipoise, in some embodiments less than about 2×10⁵ centipoise, in some embodiments less than about 2×10⁴ centipoise, and in some embodiments, less than about 2×10³ centipoise, such as measured with a Brookfield viscometer, type DV-I or LV-IV, at 60 rpm and 20° C. If desired, thickeners or other viscosity modifiers may be employed in the coating formulation to increase or decrease viscosity.

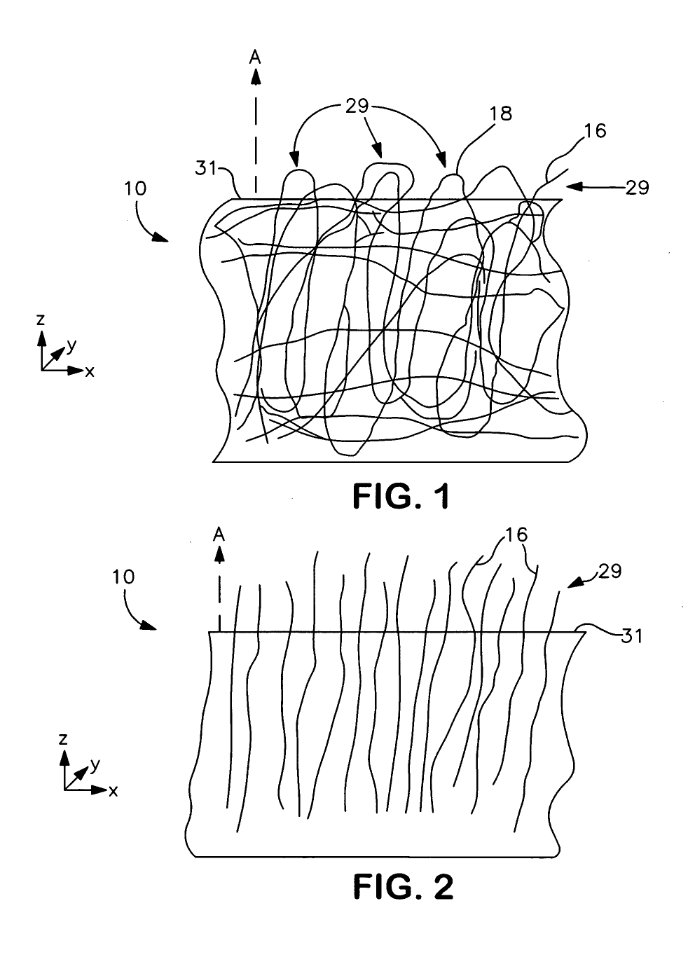

As stated above, the substrate to which the odor control coating is applied generally has a plurality of fibers oriented in the z-direction. Further, at least a portion of such fibers are exposed on the outer surface of the substrate. In accordance with the present invention, the odor control coating is applied to the substrate so that the majority (e.g., about 50 wt. %) of the coating resides on these exposed portions. Referring to FIG. 1, for instance, one embodiment of a substrate 10 is shown that contains a plurality of fibers 29 oriented primarily in the z-direction. That is, the fibers 29 are oriented at angles ranging from about 30° to about 150° relative to an axis “A”, which is perpendicular to the plane formed by the x-axis (machine direction) and y-axis (cross-machine direction). Also, at least some of the fibers 29 oriented in the z-direction have a portion that is exposed on a surface 31 of the substrate 10. That is, end portions 16 of some of the fibers 29 are exposed on the surface 31, while bent portions 18 of other fibers 29 are exposed. Likewise, referring to FIG. 2, another embodiment of the substrate 10 is shown that also contains a plurality of fibers 29 oriented primarily in the z-direction. In this particular embodiment, the fibers 29 are oriented approximately 90° relative to the axis “A”, and have end portions 16 exposed on the surface 31 of the substrate 10.

Regardless, a majority of the odor control coating (not shown) is applied to the exposed portions 16 and/or 18. By containing a majority of the odor control coating on the exposed portions of the substrate 10, any adverse affect the coating might otherwise have on certain properties of the substrate 10, such as its flexibility, absorbency, and so forth, is limited. For example, 75 wt. % or greater, and in some embodiments, 90 wt. % or greater of the odor control coating may reside on the exposed portions 16 and/or 18 of the substrate 10. Also, because the coating resides primarily on the exposed portions 16 and/or 18, other fiber portions may remain relatively free of the coating. These uncoated portions are thus more likely to retain certain desirable properties, e.g., extensibility, absorbency, etc. In addition, because the substrate 10 possesses such uncoated fiber portions, the coating may be uniformly applied, if desired, to an entire surface of the substrate. This provides an optimum surface area for the coating to contact odorous compounds during use.

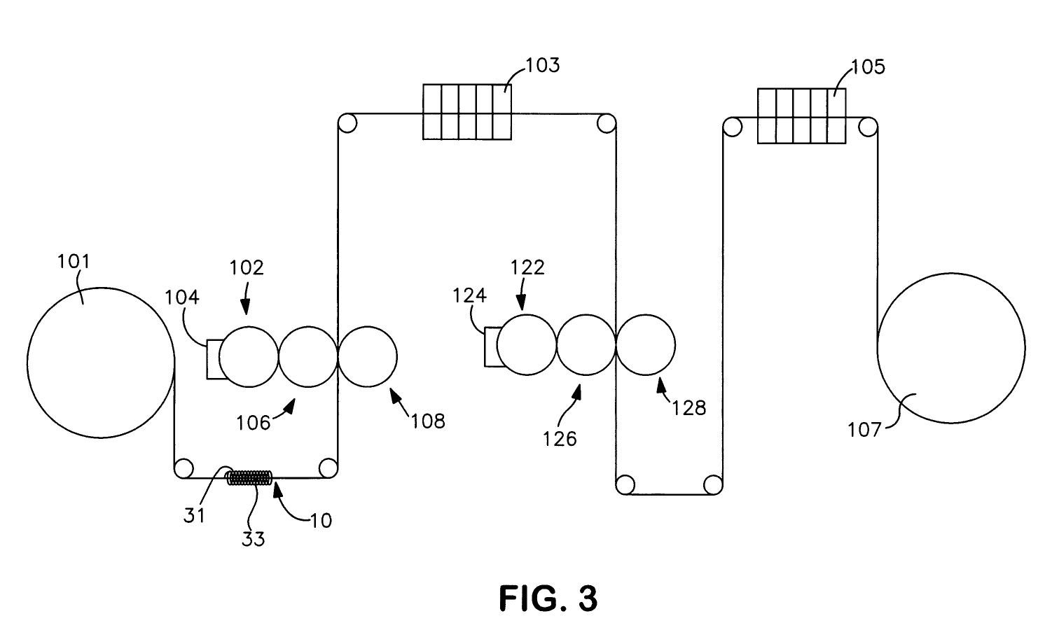

A variety of techniques may be used for applying the odor control coating in the above-described manner. Referring to FIG. 3, one embodiment of a flood coating process is illustrated that may be used to apply the odor control coating to exposed portions of the surface 31 of the substrate 10. To flood coat the surface 31, for instance, the substrate 10 is unwound from a roll 101. Alternatively, the substrate 10 may be supplied directly from a another operation (e.g. creping). A first rotatable metering roll 102 dips into a bath 104 containing the odor control coating. Upon axial rotation, the metering roll 102 acquires the odor control coating from the bath 104, wherein continuous cells (not shown) of the metering roll 102 are filled. The roll 102 then transfers the odor control coating to a transfer roll 106. The substrate 10 passes through the gap between the transfer roll 106 having the odor control coating uniformly disposed thereon and an anvil roll 108. The exposed portions on the surface 31 project toward and contact the transfer roll 106.

As the substrate 10 passes through the gap between the transfer roll 106 and the anvil roll 108, the odor control coating is applied to the exposed portions on the surface 31. The transfer roll 106 does not generally contact the remaining portions of the substrate 10, and as such, little or no odor control coating is applied thereto. Upon application, the odor control coating may be dried by a conventional dryer 103. The odor control coating may also be flood coated onto exposed portions on an opposing surface 33 of the substrate 10 using a second metering roll 122, a second bath 124, a second transfer roll 126, and a second anvil roll 128 in the manner described above. This additional odor control coating may also be dried using a dryer 105. The coated substrate 10 may then be wound up on a roll 107. Other suitable coating equipment and methods may also be described in U.S. Pat. No. 5,085,514 to Mallik, et al.; U.S. Pat. No. 5,922,406 to Ludford, III; and U.S. Pat. No. 6,299,729 to Heath, et al., which are incorporated herein in their entirety by reference thereto for all purposes.

Other techniques for coating a surface in this manner may also be utilized in the present invention. For instance, the odor control coating may be applied to a surface of the substrate using rotogravure or gravure printing, either direct or indirect (offset). Gravure printing encompasses several well-known engraving techniques, such as mechanical engraving, acid-etch engraving, electronic engraving and ceramic laser engraving. Such printing techniques provide excellent control of the composition distribution and transfer rate. Gravure printing may provide, for example, from about 10 to about 1000 deposits per lineal inch of surface, or from about 100 to about 1,000,000 deposits per square inch. Each deposit results from an individual cell on a printing roll, so that the density of the deposits corresponds to the density of the cells. A suitable electronic engraved example for a primary delivery zone is about 200 deposits per lineal inch of surface, or about 40,000 deposits per square inch. By providing such a large number of small deposits, the uniformity of the deposit distribution may be enhanced. Also, because of the large number of small deposits applied to the surface of the substrate, the deposits more readily resolidify on the exposed fiber portions. Suitable gravure printing techniques are also described in U.S. Pat. No. 6,231,719 to Garvey, et al., which is incorporated herein in its entirety by reference thereto for all purposes. Moreover, besides gravure printing, it should be understood that other printing techniques, such as flexographic printing, may also be used to apply the coating.

Still another suitable contact printing technique that may be utilized in the present invention is “screen printing.” Screen printing is performed manually or photomechanically. The screens may include a silk or nylon fabric mesh with, for instance, from about 40 to about 120 openings per lineal centimeter. The screen material is attached to a frame and stretched to provide a smooth surface. The stencil is applied to the bottom side of the screen, i.e., the side in contact with the substrate upon which the fluidic channels are to be printed. The coating is painted onto the screen, and transferred by rubbing the screen (which is in contact with the substrate) with a squeegee.

Ink-jet printing techniques may also be employed in the present invention. Ink-jet printing is a non-contact printing technique that involves forcing the ink through a tiny nozzle (or a series of nozzles) to form droplets that are directed toward the substrate. Two techniques are generally utilized, i.e., “DOD” (Drop-OnDemand) or “continuous” ink-jet printing. In continuous systems, ink is emitted in a continuous stream under pressure through at least one orifice or nozzle. The stream is perturbed by a pressurization actuator to break the stream into droplets at a fixed distance from the orifice. DOD systems, on the other hand, use a pressurization actuator at each orifice to break the ink into droplets. The pressurization actuator in each system may be a piezoelectric crystal, an acoustic device, a thermal device, etc. The selection of the type of ink jet system varies on the type of material to be printed from the print head. For example, conductive materials are sometimes required for continuous systems because the droplets are deflected electrostatically. Thus, when the sample channel is formed from a dielectric material, DOD printing techniques may be more desirable.

In addition to the printing techniques mentioned above, any other suitable application technique may be used in the present invention. For example, the odor control coating may also be sprayed onto the substrate. Any equipment suitable for spraying an additive onto a substrate may be utilized in the present invention. For instance, one example of suitable spraying equipment includes external mix, air atomizing nozzles, such as the 2 mm nozzle available from V.I.B. Systems, Inc., Tucker, Ga. Another nozzle that can be used is an H ⅛″ VV-SS 650017 VeeJet spray nozzle available from Spraying Systems, Inc. of Milwaukee, Wis. Still other spraying techniques and equipment are described in U.S. Pat. No. 5,164,046 to Ampulski, et al., which is incorporated herein in its entirety by reference thereto for all purposes. Likewise, a brush spray application technique may also be employed, such as described in U.S. Pat. No. 5,628,788 to Garavaglia, et al., which is incorporated herein in its entirety by reference thereto for all purposes. Besides the above-mentioned techniques, the odor control coating may also be applied as a foam composition. For instance, several suitable techniques for forming a foam composition and applying the composition to a dry web are described in U.S. Pat. No. 6,607,783 to Vander Heiden, et al. and U.S. Pat. No. 6,797,116 to Capizzi, which are incorporated herein in their entirety by reference thereto for all purposes. Still other suitable coating techniques may include, for instance, bar, roll, knife, curtain, slot-die, dip-coating, drop-coating, extrusion, etc.

As stated above, uniform application of the odor control is desirable in many cases to provide an optimum surface area for contacting odorous compounds. However, to further enhance extensibility, absorbency, and/or some other characteristic of the substrate, it is sometimes desired to apply the odor control coating so that it covers less than 100%, in some embodiments from about 10% to about 80%, and in some embodiments, from about 20% to about 60% of the area of one or more surfaces of the substrate. For instance, in one particular embodiment, the odor control coating is applied to the substrate in a preselected pattern (e.g., reticular pattern, diamond-shaped grid, dots, and so forth). Although not required, such a patterned coating may provide sufficient odor control without covering a substantial portion of the surface area of the substrate. This may be desired to optimize flexibility, absorbency, or other characteristics of the substrate. In addition, a patterned coating may also provide different functionality to each zone. For example, in one embodiment, the substrate is treated with two or more patterns of coated regions that may or may not overlap. The regions may be on the same or different surfaces of the substrate. In one embodiment, one region of a substrate is coated with a first odor control coating, while another region is coated with a second odor control coating. Likewise, an article may contain a first coated substrate and a second coated substrate. In either case, one region or substrate may be configured to reduce one type of odor, while another region or substrate may be configured to reduce another type of odor. Alternatively, one region or substrate may possess a higher level of an odor control coating than another region or substrate to provide different levels of odor reduction.

Regardless of the method of application, the odor control substrate may sometimes be dried at a certain temperature to drive the solvent from the coating. For example, the substrate may be heated to a temperature of at least about 50° C., in some embodiments at least about 70° C., and in some embodiments, at least about 80° C. By minimizing the amount of solvent in the coating, a larger surface area of odor adsorbent may be available for contacting odorous compounds, thereby enhancing odor reduction. It should be understood, however, that relatively small amounts of solvent may still be present. For example, the dried coating may contain a solvent in an amount less than about 10% by weight, in some embodiments less than about 5% by weight, and in some embodiments, less than about 1% by weight.

When dried, the relative percentages and solids add-on level of the resulting odor control coating may vary to achieve the desired level of odor control. The “solids add-on level” is determined by subtracting the weight of the untreated substrate from the weight of the treated substrate (after drying), dividing this calculated weight by the weight of the untreated substrate, and then multiplying by 100%. One particular benefit of the present invention is that high solids add-on levels and odor adsorbent levels are achievable without a substantial sacrifice in durability of the coating. In some embodiments, for example, the add-on level of the coating is at least about 2%, in some embodiments from about 4% to about 20%, and in some embodiments, from about 6% to about 15%. Further, the dried coating may contain from about 10 wt. % to about 80 wt. %, in some embodiments from about 20 wt. % from about 70 wt. %, and in some embodiments, from about 40 wt. % to about 60 wt. % of the odor adsorbent. Likewise, the dried coating may also contain from about 10 wt. % to about 80 wt. %, in some embodiments from about 10 wt. % from about 60 wt. %, and in some embodiments, from about 30 wt. % to about 50 wt. % of binder.

Besides having various functional benefits, the coated substrate may also have aesthetic benefits as well. For example, although sometimes containing activated carbon, the coated substrate may be made without the black color commonly associated with activated carbon. In one embodiment, as described above, white or light-colored particles (e.g., calcium carbonate, titanium dioxide, etc.) are employed in the odor control coating so that the resulting substrate has a grayish or bluish color. In addition, various pigments and/or dyes may be employed to alter the color of the odor control coating. Likewise, the pigments, dyes, or other masking agents may be applied to the substrate separately from the odor control coating. In such cases, the masking agent may be applied below and/or above the odor control coating, or applied to the substrate in regions in which the odor control coating is not applied.

D. Articles

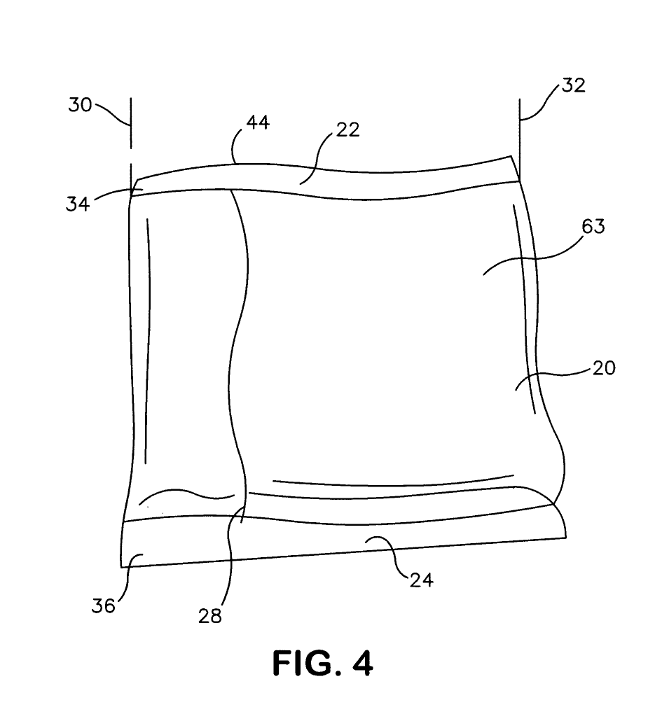

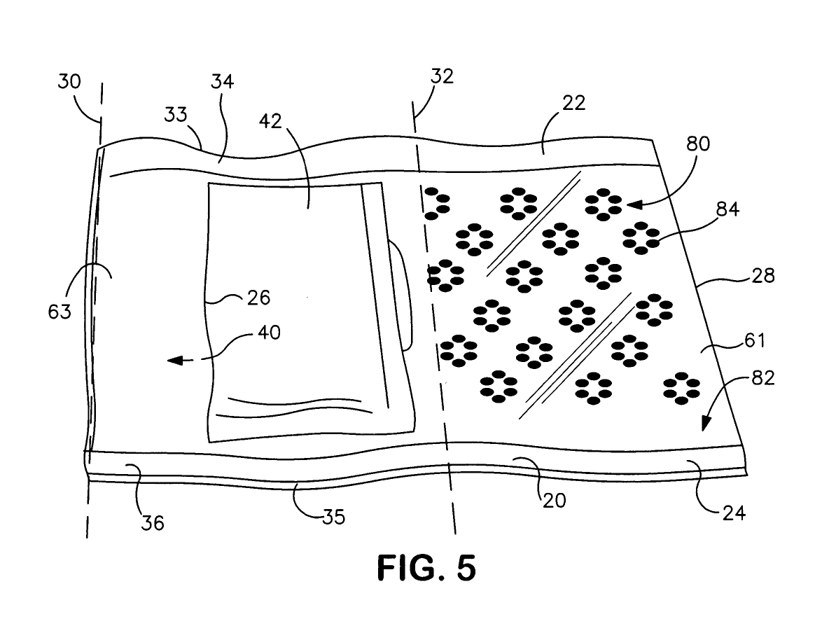

The odor control substrate of the present invention may be employed in a wide range of articles. In one particular embodiment, the odor control substrate is used to form a pouch for an absorbent article. Specifically, many absorbent articles (e.g., feminine hygiene products) are disposed by placing them in a small pouch in which the product is packaged for sale. Thus, the odor control substrate of the present invention may be employed in the pouch to help reduce odors associated with the dispensed absorbent articles. Referring to FIGS. 4-5, for example, one embodiment of an individually wrapped absorbent article package 50 is illustrated. As shown, an absorbent article 42 is carried in the package 50, which for purposes of description only, is shown as a feminine care product (e.g., sanitary pad or napkin). The absorbent article 42 may be folded in any desired pattern to fit in the package 50.

The package 50 includes an elongate piece of wrapper 44 that is folded and bonded into the desired pouch configuration. For example, the wrapper 44 may be an elongated rectangular piece having a first end 26, an opposite second end 28, and generally parallel longitudinal sides 33 and 35 extending between the ends 26 and 28. Various other pouch configurations are known and used in the art for individually packaging feminine care absorbent articles and any such configuration may be used in a package according to the invention. For example, various other pouch configurations are disclosed in U.S. Pat. No. 6,716,203 to Sorebo, et al. and U.S. Pat. No. 6,380,445 to Moder, et al., as well as U.S. Patent Application Publication No. 2003/0116462 to Sorebo, et al., all of which are incorporated herein in their entirety by reference thereto for all purposes. In the illustrated embodiment, for example, a pouch 40 is shown that is similar to the pouch configuration used for Kotex® Ultrathin pads available from Kimberly-Clark Corporation.

The wrapper 44 is essentially folded around the absorbent article 42 such that the pouch 40 is formed around the article. The wrapper 44 is first folded at a first fold axis 30 such that the first end 26 is folded towards but spaced from the second end 28. The distance between the first end 26 and second end 28 may vary depending on the desired length of a resulting flap 20, as described below. The aligned longitudinal sides of the wrapper 44 define sides 34 and 36 of the pouch 40. The second end 28 of the wrapper 44 is then folded at a second fold axis 32 so as to extend back over the first end 26 and thus defines the flap 20 that closes off the pouch 40. The flap 20 has longitudinal sides 24 and 22 that align with the material sides 33 and 35 and pouch sides 34 and 36. The sides of the pouch 40 are then bonded in a conventional manner, for example with a heat/pressure embossing roll. The flap sides 22 and 24 are bonded to the material sides 33 and 35 and pouch sides 34 and 36 in a single pass operation. It may be the case that the first end 26 of the wrapper 44 extends essentially to the second fold axis 32 and, thus, the flap sides 22 and 24 would be bonded along their entire length to pouch sides 34 and 36. The edge of the second end 28 may extend across the front surface of the pouch 40. It may be desired to adhere all or a portion of this edge to the pouch surface. However, in a desirable embodiment, this edge is left un-adhered to the pouch between its bonded sides 22 and 24.

Regardless of the particular pouch configuration, the wrapper 44 may be formed from a variety of different materials, including a film, a fibrous material (e.g., nonwoven web), an elastic material, and so forth. For example, the wrapper 44 may sometimes contain an extensible and/or elastic substrate containing the odor control coating of the present invention. As shown, for example, a pattern 80 of an odor control coating 84 may reside on an inner surface 61 of the wrapper 44 so that it is more readily able to contact odorous compounds stemming from the absorbent article 42. A coating may also be present on other surfaces of the wrapper 44, such as an outer surface 63. Regardless, when applied in accordance with the present invention, the substrate may remain extensible and/or elastic to provide various benefits to the resulting pouch. For example, the extensible and/or elastic substrate may allow the pouch to expand and fit over a pad or diaper that is swollen due to the absorbance of liquids during use. In addition, the coated substrate is also capable of reducing odor.

The odor control coating of the present invention may also be used in one or more components of the absorbent article itself, such as in a liquid-permeable layer (e.g., bodyside liner, surge layer, etc.), liquid-impermeable or breathable layer (e.g., outer cover, ventilation layer, baffle, etc.), absorbent core, elastic member, and so forth. Several examples of such absorbent articles are described in U.S. Pat. No. 5,197,959 to Buell; U.S. Pat. No. 5,085,654 to Buell; U.S. Pat. No. 5,634,916 to Lavon, et al.; U.S. Pat. No. 5,569,234 to Buell, et al.; U.S. Pat. No. 5,716,349 to Taylor, et al.; U.S. Pat. No. 4,950,264 to Osborn, III; U.S. Pat. No. 5,009,653 to Osborn, III; U.S. Pat. No. 5,509,914 to Osborn, III; U.S. Pat. No. 5,649,916 to DiPalma, et al.; U.S. Pat. No. 5,267,992 to Van Tillburg; U.S. Pat. No. 4,687,478 to Van Tillburg; U.S. Pat. No. 4,285,343 to McNair; U.S. Pat. No. 4,608,047 to Mattingly; U.S. Pat. No. 5,342,342 to Kitaoka; U.S. Pat. No. 5,190,563 to Herron, et al.; U.S. Pat. No. 5,702,378 to Widlund, et al.; U.S. Pat. No. 5,308,346 to Sneller, et al.; U.S. Pat. No. 6,110,158 to Kielpikowski; U.S. Pat. No. 6,663,611 to Blaney, et al.; and WO 99/00093 to Patterson, et al., which are incorporated herein in their entirety by reference thereto for all purposes. In one embodiment, for example, the odor control substrate is employed as a surge layer in an absorbent article that helps to decelerate and diffuse surges or gushes of liquid that may be rapidly introduced into the absorbent core. The surge layer rapidly accepts and temporarily holds the liquid prior to releasing it into the storage or retention portions of the absorbent core. The surge layer is typically constructed from highly liquid-permeable materials. Examples of suitable surge layers are described in U.S. Pat. No. 5,486,166 to Ellis, et al. and U.S. Pat. No. 5,490,846 to Ellis, et al., which are incorporated herein in their entirety by reference thereto for all purposes.

The odor control coating of the present invention is versatile and may also be used with other types of articles of manufacture. For instance, the odor control coating may be used in air filters, such as house filters, vent filters, disposable facemasks, and facemask filters. Exemplary facemasks, for instance, are described and shown, for example, in U.S. Pat. Nos. 4,802,473; 4,969,457; 5,322,061; 5,383,450; 5,553,608; 5,020,533; 5,813,398; and 6,427,693, which are incorporated herein in their entirety by reference thereto for all purposes. In one embodiment, a substrate coated with the odor control coating of the present invention may be utilized as a filtration layer of the facemask. Filtration layers, such as meltblown nonwoven webs, spunbond nonwoven webs, and laminates thereof, are well known in the art.

In still other embodiments, the odor control coating may be employed in conjunction with a garment. For instance, garments, such as meat and seafood packing industry aprons/attire, grocery store aprons, paper mill aprons/attire, farm/dairy garments, hunting garments, etc., may be incorporated with the odor control coating of the present invention. As an example, hunters often wear garments that are camouflaged for the particular hunting environment. The odor control coating of the present invention may thus be used to form the camouflage pattern. Specifically, the odor control coating may impart the desired color pattern and also help reduce human odor during hunting. In addition, the odor control coating may be employed on a cover for pet beds, chairs, elder care/hospital bed covers, infant/children cribs, and so forth.

The present invention may be better understood with reference to the following examples.

### EXAMPLE 1

The ability to apply an odor control coating to a substrate in accordance with the present invention was demonstrated. Initially, a 6″×12″ fabric sample was provided for coating. The sample contained a polypropylene spunbond web (basis weight of approximately 0.5 ounces per square yard) laminated to a polypropylene film (pre-stretched 80%). The laminate was also applied with a blend of bicomponent fibers (1% by weight of the fabric) and polyester fibers (1% by weight of the fabric) by extruding the fibers from a spinnerette and then thermally bonding them to the spunbond/film laminate. The bicomponent fibers were obtained from Fibervisions, Inc. of Covington, Ga. under the name “ESC 215”, which had a polyethylene sheath and polypropylene core, a denier of 1.5, and 0.55 wt. % “HR6” finish. The polyester fibers were obtained from Invista of Wichita, Kans. under the name “T-295”, which had a denier of 6.0 and contained a 0.5 wt. % L1 finish.

An activated carbon ink was also provided that was obtained from MeadWestvaco Corp. under the name “Nuchar PMA”, and contained 15 wt. % activated carbon, 12 wt. % styrene-acrylic copolymer binder, and 73 wt. % water. The activated carbon ink was applied to the fabric sample using a Myer rod. Specifically, the fabric sample was placed on the bench top and held in place using strips of masking tape. 10 milliliters of the carbon ink was deposited on the bench just ahead of the fabric top to form a line of liquid. A Myer rod (No. 10) was then placed ahead of the ink line and drawn towards the fabric, over the top of the fabric, and then off the fabric at the bottom of the sample. This was done to deposit a coating of the ink on the fabric without stopping or leaving a pool of the ink on its surface. After applying the coating, it was dried in a convection oven at 80° C. for 4 minutes.

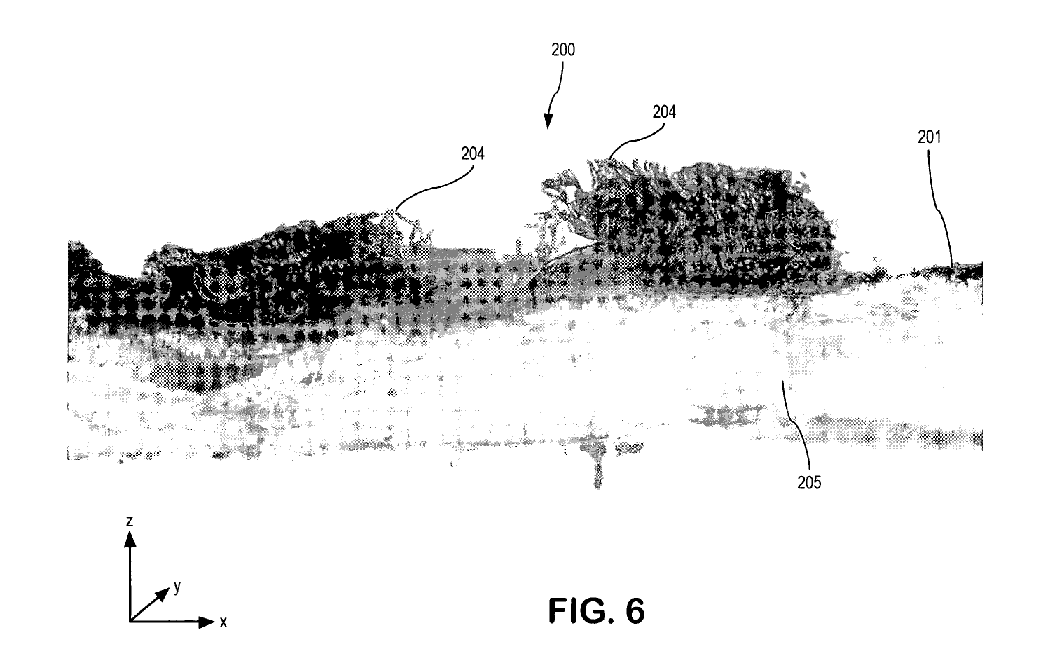

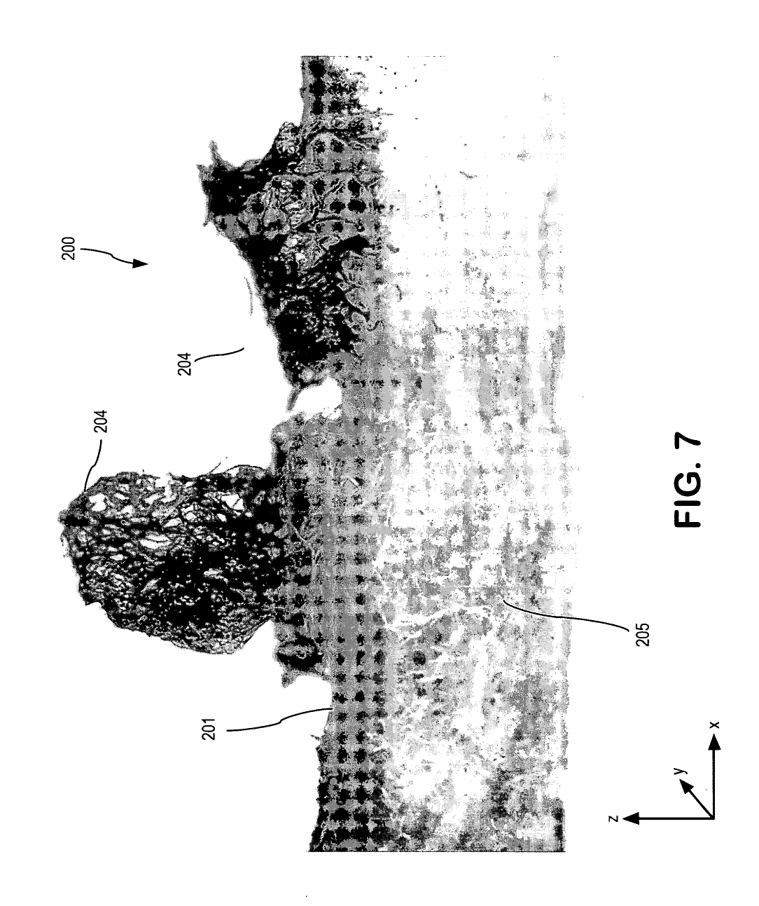

The resulting solids add-on level (dry) was 5%. The sample remained extensible, and no rub-off or loss of coating was observed upon stretching and releasing. In addition, cross-sectional photographs were also taken of the coated fabric at different locations, which are provided as FIGS. 6 and 7. As indicated, the fabric sample 200 contained z-directional fibers 204 exposed on a surface 201. The exposed fibers 204 contained a majority of the activated carbon ink, which is shown as having a dark black color. Likewise, little if any of the coating is shown to be present on fibers 205 oriented primarily in the x-y plane of the sample 200.

### EXAMPLE 2

An activated carbon ink was coated onto a fabric as described in Example 1, except that the ink was applied to the substrate using a steel gravure printing roll. 50 milliliters of the carbon ink was placed in a Pyrex glass pan. The gravure roll was rolled up and down in the pan of ink to coat the roller. The coated roller was then placed onto the fabric and rolled down the sample to deposit the ink onto the top fibers of the fabric. The coated substrate was dried in a convection oven at 85° C. for 5 minutes. The resulting solids add-on level (dry) was 10%. The sample remained extensible, and no rub-off or loss of coating was observed upon stretching and releasing.

### EXAMPLE 3

An activated carbon ink was coated onto a fabric as described in Example 1, except that the ink was applied to the substrate using a rubber printing roll obtained from Michaels, Inc. under the name “Speedball.” 50 milliliters of the carbon ink was placed in a Pyrex glass pan. The rubber roll was rolled up and down in the pan of ink to coat the roller. The coated roller was then rolled across the sample to deposit a thin coating of the ink onto the top fibers of the fabric. The coated substrate was dried in a convection oven at 85° C. for 5 minutes. The resulting solids add-on level (dry) was 0.8%. The sample remained extensible, and no rub-off or loss of coating was observed upon stretching and releasing.

### EXAMPLE 4

An activated carbon ink was coated onto a fabric as described in Example 1, except that the ink was applied to the substrate using a painter foam applicator brush obtained from Home Depot. 50 milliliters of the carbon ink was placed in a 100-milliliter glass beaker. The brush was dipped into the ink and painted onto the fabric. The coated substrate was dried in a convection oven at 85° C. for 5 minutes. The resulting solids add-on level (dry) was 9%. The sample remained extensible, and no rub-off or loss of coating was observed upon stretching and releasing.

### EXAMPLE 5

An activated carbon ink was coated onto a fabric as described in Example 1, except that the ink was applied to the substrate using a brush technique. Specifically, a bristled roller having a width of 6 inches and a diameter of 2 inches was employed. 100 milliliters of carbon ink was placed into a Pyrex glass dish. The roller was placed into the ink, and rolled back and forth to coat the roller. The fabric was then coated by rolling the roller across the surface of the fabric sample. The coated substrate was then dried in a convection oven at 85° C. for 5 minutes. The resulting solids add-on level (dry) was 8%. The substrate remained extensible, and no rub-off or loss of coating was observed upon stretching and releasing.

### EXAMPLE 6

The ability to apply an odor control coating to a substrate in accordance with the present invention was demonstrated. Initially, various fabric samples were provided for coating. The fabric samples were a spunbond/meltblown/spunbond (SMS) laminate. The spunbond webs were formed from polypropylene and had a basis weight of 1 ounce per square yard. The meltblown web was formed from an S-EB-S block copolymer (Kraton Polymers) and had a basis weight of 0.4 ounces per square yard. An activated carbon ink was also provided that was obtained from MeadWestvaco Corp. under the name “Nuchar PMA”, and contained 15 wt. % activated carbon, 12 wt. % styrene-acrylic copolymer binder, and 73 wt. % water. The activated carbon ink was applied to the fabric samples using a Myer rod as described in Example 1. After applying the coating, it was dried in a convention oven at 85° C. for 5 minutes. The resulting solids add-on level (dry) was 9%. The samples remained extensible, and no rub-off or loss of coating was observed upon stretching and releasing.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. An odor control substrate having a plane of orientation defined by the cross machine direction and machine direction of the substrate, the substrate comprising a plurality of fibers oriented from about 30° to about 150° relative to an axis that is perpendicular to the plane of orientation, at least a portion of said fibers being exposed on a surface of the substrate, wherein an odor control coating is applied to the substrate so that a majority of said coating resides on said exposed fibers, said odor control coating comprising an odor adsorbent that is capable of adsorbing one or more odorous compounds when contacted therewith.

2. The odor control substrate of claim 1, wherein said fibers are oriented from about 60° to about 120° relative to an axis that is perpendicular to the plane of orientation of the substrate.

3. The odor control substrate of claim 1, wherein the substrate comprises a nonwoven web.

4. The odor control substrate of claim 1, wherein said fibers are crimped, multicomponent fibers.

5. The odor control substrate of claim 1, wherein the substrate is extensible in one or more directions.

6. The odor control substrate of claim 1, wherein the substrate comprises an elastic material.

7. The odor control substrate of claim 1, wherein said odor adsorbent is selected from the group consisting of activated carbon, zeolites, silica, clays, alumina, magnesia, titania, cyclodextrins, and combinations thereof.

8. The odor control substrate of claim 7, wherein said odor adsorbent is activated carbon.

9. The odor control substrate of claim 1, wherein said odor adsorbent constitutes from about 10 wt. % to about 80 wt. % of said coating.

10. The odor control substrate of claim 1, wherein said odor adsorbent constitutes from about 40 wt. % to about 60 wt. % of said coating.

11. The odor control substrate of claim 1, wherein said coating comprises a binder.

12. The odor control substrate of claim 11, wherein said binder constitutes from about 10 wt. % to about 80 wt. % of said coating.

13. The odor control substrate of claim 1, wherein the solids add-on level of said coating is at least about 2%.

14. The odor control substrate of claim 1, wherein at least about 75 wt. % of said coating resides on said exposed fibers.

15. The odor control substrate of claim 1, wherein at least about 90 wt. % of said coating resides on said exposed fibers.

16. A pouch for an absorbent article, the pouch comprising the substrate of claim 1.

17. An absorbent article comprising the substrate of claim 1.

18. A method for forming an odor control substrate, said method comprising:
providing a substrate having a plane of orientation defined by the cross machine direction and machine direction of the substrate, the substrate comprising a plurality of fibers oriented from about 60° to about 120° relative to an axis that is perpendicular to the plane of orientation, at least a portion of said fibers being exposed on a surface of the substrate;
forming a coating formulation that comprises activated carbon and a solvent;
coating the surface of the substrate with said formulation; and
drying said formulation to form an odor control coating that is present on said exposed fibers.

19. The method of claim 18, wherein said coating formulation is flood coated onto said substrate.

20. The method of claim 18, wherein said coating formulation is printed onto said substrate.

21. The method of claim 18, further comprising coating another surface of the substrate with a coating formulation that comprises activated carbon.

22. The method of claim 18, wherein said solvent comprises water.

23. The method of claim 18, wherein said solvent constitutes from about 40 wt. % to about 99 wt. % of said coating formulation.

24. The method of claim 18, wherein said coating formulation further comprises a binder.

25. The method of claim 24, wherein said binder constitutes from about 0.01 wt. % to about 30 wt. % of said coating formulation.

26. The method of claim 18, wherein said activated carbon comprises from about 1 wt. % to about 50 wt. % of said coating formulation.

27. The method of claim 18, wherein the substrate comprises a nonwoven web.

28. The method of claim 18, wherein the substrate is extensible in one or more directions.

29. The method of claim 18, wherein a majority of said coating resides on said exposed fibers.

30. The method of claim 18, wherein at least about 75 wt. % of said coating resides on said exposed fibers.

31. The method of claim 18, wherein at least about 90 wt. % of said coating resides on said exposed fibers.

32. A pouch for individually wrapping a feminine care absorbent article, the pouch comprising a wrapper that contains a nonwoven fibrous material, said material having a plane of orientation defined by the cross machine direction and machine direction of said material, said nonwoven fibrous material comprising a plurality of fibers oriented from about 30° to about 150° relative to an axis that is perpendicular to the plane of orientation, at least a portion of said fibers being exposed on a surface of said material, wherein an odor control coating is applied to said nonwoven fibrous material so that a majority of said coating resides on said exposed fibers.

33. The pouch of claim 32, wherein said fibers are oriented from about 60° to about 120° relative to an axis that is perpendicular to the plane of orientation of said nonwoven fibrous material.

34. The pouch of claim 32, wherein said nonwoven fibrous material is extensible in one or more directions.

35. The pouch of claim 32, wherein said nonwoven fibrous material comprises an elastic material.

36. The pouch of claim 32, wherein said odor control coating comprises activated carbon.
